(19) United States
(12) Patent Application Publication
(10) Pub. No.: US 2005/0075277 A1
(43) Pub. Date: Apr. 7, 2005

(54) Use of lactoferrin in prophylaxis against infection and/or inflammation in immunosuppressed subjects

(75) Inventors: Atul Varadhachary, Houston, TX (US); Rick Barsky, Houston, TX (US); Ernest Yankee, Houston, TX (US)

Correspondence Address: FULBRIGHT & JAWORSKI, LLP, 1301 MCKINNEY, SUITE 5100, HOUSTON, TX, 77010-3095, US

(73) Assignee: AGENNIX INCORPORATED

(21) Appl. No.: 10/889,539

(22) Filed: Jul. 12, 2004

Related U.S. Application Data
(60) Provisional application No. 60/486,100, filed on Jul. 10, 2003.

(51) Int. Cl. A61K 38/40; A61K 31/56

(52) U.S. Cl. 514/6; 514/171

(57) ABSTRACT

The present invention relates to a use of lactoferrin in prophylaxis against infection and/or inflammation in immunosuppressed subjects or subjects whose immune systems are expected to be suppressed. Specifically, the invention provides a method of preventing infection and/or inflammation in individuals by administrating an effective amount of pharmaceutical formulation comprised of a lactoferrin product.

This application claims priority to U.S. Provisional Application No. 60/486,100 filed Jul. 10, 2003, which is incorporated herein in its entirety.

### TECHNICAL FIELD

This invention relates generally to methods and pharmaceutical compositions for prophylactic treatment of infections and/or inflammations in immunosuppressed subjects or subjects whose immune systems are expected to be suppressed. Specifically, the invention provides a method of preventing mucositis in individuals undergoing therapies that typically cause neutropenia.

### BACKGROUND OF THE INVENTION

In the last decades, the incidence of severe and systemic infections has increased dramatically because of the growing number of immunosuppressed patients suffering from AIDS, diabetes, cancer and other conditions. In addition, the widespread use of immunosuppressants for organ transplant patients, the common practice of radiation and chemotherapy for treating malignancies, as well as the growing size of the aging population have increased the morbidity of opportunistic and common pathogens.

In particular, cancer patients undergoing aggressive chemotherapy and radiotherapy face an enhanced risk of infections and/or inflammation due to the frequent development of neutropenia. One common type of inflammation is oral mucositis. Although there is substantial variability between individual patients, almost all of the anticancer drugs will cause mucositis if a large enough dose is given. Early signs of mucositis include mild erythema and edema of the bucccal mucosa. This generalized inflammation may progress to painful ulceration and secondary infections. Mouth sores usually occur 2 to 14 days after a dose of chemotherapy and take at least 7 days to heal after chemotherapy is discontinued. Mucositis may lead to complications such as malnutrition (i.e., it may be too painful to eat), dehydration (i.e., pain on swallowing may prevent all oral intake), bleeding may be a sign of problems (such as thrombocytopenia), infection, and refusal or interruption of therapy.

Current treatment for mucositis is mainly palliative and not therapeutic, consisting of maintaining good oral hygiene and providing temporary pain relief. These procedures are of limited value, because they are only palliative and not prophylactic. Thus, there remains an unmet medical need to develop an effective regimen to minimize these complications especially in immunosuppressed individuals.

Lactoferrin is a member of the transferrin family of non-heme iron binding proteins and is normally found in serum and exocrine secretions such as milk, seminal fluid, intestinal secretions, tears, sweat, saliva and nasal secretions in mammals ((Metz-Boutigue et al., 1984; Masson et al., 1971; Levay et al., 1995).

Lactoferrin has been found to possess anti-infective and anti-inflammatory properties (Miehlke et al., 1996; Ward et al., 1995; Cumberbatch et al., 2000; Kimber et al., 2000; Vorland et al., 1999; Arnold R. R., et al., 1980; Bellamy W. et al., 1993; Isamida. et al., 1998; Matthews et al., 1976). Specifically, lactoferrin was found to inhibit growth of bacteria either by indirectly binding iron molecules, or by directly destabilizing the outer membranes of bacteria (Vorland, 1999). Lactoferrin and lactoferricin B (a bovine lactoferrin-derived peptide consisting of 25 amino acid residues generated by acid-pepsin hydrolysis) have been shown to inhibit the growth of fungi (Tomita, 1991; Ellison, et al., 1998). Lactoferrin has also been recognized as a potent inhibitor of various viruses (Matthews et. al.; Harsen et al., 1995; Marchetti et al., 1998; Ikeda et al., 1999).

In addition to the anti-microbial properties, lactoferrin has been found to ameliorate gastric intestinal infections and other forms of infection as well (Levay et al., 1993; Dial et al., 2002; Sato et al., 1996).

A number of clinical studies have been conducted to demonstrate these and other therapeutic potentials of both natural lactoferrin and recombinant lactoferrin. For instance, Inoue et al. have reported that orally administered natural human lactoferrin significantly reduces GVDH (graft versus host disease) of patients who have received an allogeneic bone marrow transplant and suffered from severe GVHD (Inoue et al., 1998; Inoue et al., 2001).

Studies have also been done to assess the safety of lactoferrin. Recombinant lactoferrin (rhLF) appears to be well-tolerated as demonstrated in pre-clinical and clinical studies.

Despite a wealth of clinical studies that establish the anti-microbial utility of lactoferrin, its prophylactic effect against infection and inflammation on neutropenic patients has not been thoroughly investigated. For instance, although Trumpler et al. has explored the possibility of this application (Trumpler et al., 1989), Trumpler et al. has failed to provide a specific treatment regimen that will a) prolong the infection-free interval during which no observable mucositis infection and/or inflammation occurs; and/or b) reduce both the severity and the frequency of the infection.

Thus, there exists a considerable need for prophylaxis against infections and/or inflammations, specifically for mucositis in immunosuppressed patients. Yet further, there is a need for prophylaxis of neutropenia in an immunosuppressed patient. The present invention addresses this need and provides related advantages as well.

### BRIEF SUMMARY OF THE INVENTION

A principal aspect of the present invention is a novel use of lactoferrin in the prophylaxis against infection and inflammation in immunosuppressed individuals or individuals whose immune systems are expected to be suppressed.

Accordingly, this present invention provides such a method wherein the subject is immunosuppressed, and is administered an effective amount of a pharmaceutical formulation comprised of lactoferrin.

The lactoferrin composition, which is dispersed in a pharmaceutically acceptable carrier, comprises lactoferrin or an N-terminal lactoferrin variant in which at least the N-terminal glycine residue is truncated or substituted. The lactoferrin is mammalian lactoferrin, more particularly, the lactoferrin is human or bovine. Yet further, the lactoferrin is recombinant lactoferrin. N-terminal lactoferrin variants include variants that at least lack the N-terminal glycine residue or contain a substitution at the N-terminal glycine residue. The substitution can comprise substituting a natural or artificial amino acid residue for the N-terminal glycine residue. For example, the substitution can comprise substituting a positive amino acid residue or a negative amino acid residue for the N-terminal glycine residue or substituting a neutral amino acid residue other than glycine for the N-terminal glycine residue. Other N-terminal lactoferrin variants include lactoferrin lacking one or more N-terminal residues or having one or more substitutions in the N-terminal. In specific embodiments, the N-terminal lactoferrin variant comprises at least 1% of the lactoferrin composition, at least 5% of the lactoferrin composition, at least 10% of the lactoferrin composition, at least 25% of the lactoferrin composition, at least 50% of the lactoferrin composition or any range in between.

The amount of the lactoferrin that is administered is about 1 mg to about 100 g per day, more preferably, the amount is about 10 mg to about 10 g per day.

The present invention also provides a method for preventing infection and/or inflammation in a subject who is expected to undergo a therapy that results in reduction or loss of neutrophil cells in the subject. The method involves administering to the subject an effective amount of a lactoferrin composition prior to or concurrently with the therapy. In one aspect, the method further comprises the step of increasing the amount of lactoferrin in the gastrointestinal tract of the subject. While not wishing to be bound by a single theory, it is expected that the lactoferrin product stimulates the production of interleukin-18, which in turn stimulates the production and/or activity of immune cells. The affected immune cells may be T lymphocytes (e.g. CD4+, CD8+ and CD3+ cells) or natural killer cells. It is further envisioned that the effective amount of lactoferrin stimulated the production and/or activity of neutrophils, thereby preventing the immunosuppressed subject from becoming neutropenic.

In one aspect of these embodiments, the inflammation and/or infection to be prevented is mucositis. In another aspect, the immunosuppressed subject may be infected with AIDS, receiving an immunosuppressive agent (e.g. corticosteroids), suffering from neutropenia, or any forms of cancer. Non-limiting forms of cancer include hematopoietic or solid tumor malignancy, lymphoma, myeloma, melanoma, non-small cell lung cancer, small-cell lung cancer, hepatocarcinoma, retinoblastoma, astrocytoma, gliobastoma, leukemias, neuroblastoma, squamous cell cancer, head cancer, neck cancer, gum cancer, tongue cancer, breast cancer, pancreatic cancer, prostate cancer, renal cancer, bone cancer, testicular cancer, ovarian cancer, mestohelioma, sarcoma, cervical cancer, gastrointestinal cancer, brain cancer, colon cancer, myelodysplastic syndrome, and bladder cancer.

In yet another aspect, the subject undergoes or in expected to undergo a therapy selected from the group consisting of chemotherapy, radiotherapy, myelosuppressive therapy, myeloablative therapy, and any combinations thereof. In yet another aspect, upon completion of a chemotherapy or radiotherapy, the patient may undergo peripheral stem cell or bone marrow transplant. In still yet another aspect, the bone marrow transplantation encompasses both autologous and allogeneic transplant procedure. The latter may involve either a related or an unrelated donor.

In yet another separate aspect, the subject method (a) reduces severity of the infection and/or inflammation; (b) prolongs the period during which the immunosuppressed subject exhibits no detectable infection and/or inflammation; and/or (c) reduces incidence of the infection and/or inflammation.

The lactoferrin administered can be natural or recombinant. It may be administered via any one of the following routes: oral, intravenous, intramuscular, subcutaneous, and transdermal routes. The effective amount of lactoferrin to be administered may vary but is generally within the range of about 10 mg lactoferrin/day to about 70 gram lactoferrin/day, preferably from about 0.5 gram lactoferrin/day to about 50 gram lactoferrin/day. The prophylactic treatment may last at least about 7 days, preferably at least about 20 days, more preferably at least about 30 days, and more preferably between about 20 days to about 30 days. It may also be given intermittently, at least once every seven days through the course of the immunosuppression.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows high dose challenge with Candida tropicalis.

FIG. 2 shows low dose challenge with Candida tropicalis.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure.

Before describing the present invention in detail, it is to be understood that this invention is not limited to particular drugs or drug delivery systems, as such may vary. It is also to be understood that the terminology used herein is for the purpose of describing particular embodiments only, and is not intended to be limiting.

I. Definitions

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “containing”, “including” and “comprising” are interchangeable and one of skill in the art is cognizant that these terms are open ended terms.

The term “lactoferrin” or “LF” as used herein refers to native or recombinant lactoferrin. Native lactoferrin can be obtained by purification from mammalian milk or colostrum or from other natural sources. Recombinant lactoferrin (rLF) can be made by recombinant expression or direct production in genetically altered animals, plants, fungi, bacteria, or other prokaryotic or eukaryotic species, or through chemical synthesis.

The term “lactoferrin composition” as used herein refers to a composition having lactoferrin, a portion or part of lactoferrin, an N-terminal lactoferrin variant, or a combination thereof.

The term “N-terminal lactoferrin variant” as used herein refers to lactoferrin wherein at least the N-terminal glycine has been truncated and/or substituted. N-terminal lactoferrin variants also include, but are not limited to deletion and/or substitution of one or more N-terminal amino acid residues, for example 1, 2, 3, 4, 5, 6, 7, 8, 9, 10, 11, 12, 13, 14, 15, or 16 N-terminal amino acid residues, etc. Thus, N-terminal lactoferrin variants comprise at least deletions or truncations and/or substitutions of 1 to 16 N-terminal amino acid residues. The deletion and/or substitution of at least the N-terminal glycine of lactoferrin mediates the same biological effects as full-length lactoferrin and/or may enhance lactoferrin's biological activity, for example by stimulating the production of various cytokines (e.g., IL-18, MIP-3α, GM-CSF or IFN-γ) by inhibiting various cytokines, (e.g., IL-2, IL-4, IL-5, IL-6, IL-10, and TNF-α) thus modulating neutropenia in immunosuppressed subjects or modulating infection and/or inflammation in immunosuppressed subjects or modulating mucositis and/or increasing the levels of neutrophils in the blood or the ANC.

The terms “preventing,” “prevent,” as used herein generally refer to reduction in severity and/or frequency of symptoms, prevention of the occurrence of symptoms or their underlying cause and damage.

The terms “prophylactic treatment” or “prophylactic treating” as used herein refers to preventing, inhibiting or abrogating the disease or condition. More specifically, prophylactic treatment or prophylactic comprises administering the lactoferrin composition of the present invention to the subject prior to the development of the disease or condition, for example neutropenia.

The term “modulate” as used herein refers preventing, reducing, inhibiting or abrogating the disease or condition. Thus, the term modulate or modulating or modulation includes prophylactic treatment or prophylactic treating of a subject.

The term “leukocyte” as used herein is defined as a general term for a white blood cell. Leukocytes include lymphocytes, polymorphonuclear leukocytes, and monocytes.

The term “monocyte” as used herein refers to white blood cells that circulate in the blood stream. Monocytes differentiate into macrophages upon migration into the tissues.

The term “macrophage” as used herein refers to a large mononuclear phagocytic cell that is important in innate immunity, in early non-adaptive phases of host defense, as antigen presenting cells, and as effector cells in humoral and cell-mediated immunity. Macrophages are migratory cells deriving from bone marrow precursors and are found in most tissues in the body. Macrophage activation is important in controlling infection and also causes damage to neighboring tissues.

The term “neutrophil” or “neutrophilic polymorphonuclear leukocyte” as used herein is the major class of white blood cells in peripheral blood. Neutrophils have an important role in engulfing and killing extracellular pathogens.

The term “natural killer cells” or “NK” as used herein is defined as large, usually granular non-T cell, non-B cell lymphocytes, which kill certain tumor cells. NK cells are important in innate immunity to viruses and other intracellular pathogens.

The terms “active agent,” “drug” and “pharmacologically active agent” are used interchangeably herein to refer to a chemical material or compound that induces a desired effect. In the preferred embodiment herein, the terms refer to a lactoferrin composition which is being administered into an immunosuppressed subject, or a subject expected to or is undergoing a therapy that normally results in neutropenia. Included are derivatives and analogs of those products specifically mentioned herein or known in the art which also induce the desired effect.

The term “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The term “an effective amount” refers to the amount of a drug or pharmacologically active agent or pharmaceutical formulation that is nontoxic but is sufficient amount of the drug, agent or formulation to provide the desired effect, i.e., modulation of infection or inflammation, a reduction in infection or inflammation or prophylaxis against infection, for example mucositis in immunosuppressed subjects or subjects prophylaxis of neutropenia in subjects that are expected to suffer from neutropenia as a result of therapy.

The term “prophylactic amount” refers to an amount that results in prophylaxis against infection, for example mucositis in immunosuppressed subjects or subjects prophylaxis of neutropenia in subjects that are expected to suffer from neutropenia as a result of therapy.

The term “oral administration” as used herein includes oral, buccal, enteral or intragastric administration.

The term “parenteral administration” as used herein includes any form of administration in which the compound is absorbed into the subject without involving absorption via the intestines. Exemplary parenteral administrations that are used in the present invention include, but are not limited to intramuscular, intravenous, intraperitoneal, intraocular, subcutaneous or intraarticular administration. Yet further, parenteral administration also includes administration into a surgical field.

The term “subject” as used herein, is taken to mean any mammalian subject to which a lactoferrin composition is administered according to the methods described herein. Thus, a skilled artisan realizes that a mammalian subject, includes, but is not limited to humans, monkeys, horses, pigs, cows, dogs, cats, rats and mice. In a specific embodiment, the methods of the present invention are employed to treat a human subject.

The term “topical administration” as used herein includes, but is not limited to topical, dermal, epidermal, oro-pharyngeal cavity, vaginal, rectal, or perineum.

The term “immunosuppressed” as used herein refers to a subject is an individual whose immune system has been compromised, possibly due to the infection of bacteria, fungi, viruses, and/or protozoa, or due to the use of drugs that suppress the functions of the immune system.

The term “infection” refers to an infectious condition cause by bacteria, fingi, viruses, protozoa, or any combinations thereof.

The term “mucositis” refers to an inflammatory response of the bodily mucosa and surrounding soft tissues. Common forms of mucositis include but are not limited to oral mucositis and gastric intestinal mucositis.

The term “cancer patient” is defined as a patient suffering from any cancer or neoplasm or malignant tumors found in mammals. Non-limiting forms of cancer include hematopoietic or solid tumor malignancy, lymphoma, myeloma, melanoma, non-small cell lung cancer, small-cell lung cancer, hepatocarcinoma, retinoblastoma, astrocytoma, gliobastoma, leukemias, neuroblastoma, squamous cell cancer, head cancer, neck cancer, gum cancer, tongue cancer, breast cancer, pancreatic cancer, prostate cancer, renal cancer, bone cancer, testicular cancer, ovarian cancer, mesothelioma, sarcoma, cervical cancer, gastrointestinal cancer, brain cancer, colon cancer, myelodysplastic syndrome, and bladder cancer.

The term “neutropenia” as used herein refers to an a decrease or small number of neutrophils in the blood compared to normal. For example, the World Health Organization defines neutropenia as a subject having an absolute neutrophil cell count (ANC) of about 2000 cells/μL or less. Thus, as used herein a subject suffering from neutropenia is one having an ANC of about 2000 cells/μL or less, for example 1000 cells/μL or even less than 500 cells/μL.

The term “Grade 3 neutropenia” refers to the reduction of the absolute neutrophil cell count (ANC) to less than about 1000 cells/μL. The term “neutropenia” generally refers to a condition in which the ANC is reduced to 1000 cells//μL or less. Such a condition may be caused by depressed production, increased peripheral destruction of neutrophils. The most common neutropenias are iatrogenic, resulting from the widespread use of cytotoxic or immunosuppressive therapies for cancer treatment or control of autoimmune disorders. Other causes of neutropenia include induction by drugs, hematological diseases including idiopathic, cyclic neutropenia, Chediak-Higashi syndrome, aplastic anemia, infantile genetic disorders, tumor invasion such as myelofibrosis, nutritional deficiency; infections such as tuberculosis, typhoid fever, brucelloisis, tularemia, measles, infectious mononucleosis, malaria, viral hepatitis, leishmaniasis, AIDS, antineutrophil antibodies and/or splenetic or lung trapping, autoimmune disorders, wegner's granulomatosis, acute endotoxemia, hemodialysis, and cardiopulmonary bypass. The present invention applies to any acquired and inherited neutropenic conditions.

II. Lactoferrin

The lactoferrin used according to the present invention can be obtained through isolation and purification from natural sources, for example, but not limited to mammalian milk. The lactoferrin is preferably mammalian lactoferrin, such as bovine or human lactoferrin. In preferred embodiments, the lactoferrin is produced recombinantly using genetic engineering techniques well known and used in the art, such as recombinant expression or direct production in genetically altered animals, plants or eukaryotes, or chemical synthesis. See, e.g., U.S. Pat. Nos. 5,571,896; 5,571,697 and 5,571,691, which are herein incorporated by reference.

In certain aspects, the present invention provides lactoferrin variants having enhanced biological activities over natural LF and or rLF, e.g., the ability to stimulate and/or inhibit cytokines or chemokines. In particular, the invention provides variants of lactoferrin from which at least the N-terminal glycine residue has been substituted and/or truncated. The N-terminal lactoferrin variants may occur naturally or may be modified by the substitution or deletion of one or more amino acids.

The deletional variants can be produced by proteolysis of lactoferrin and/or expression of a polynucleotide encoding a truncated lactoferrin as described in U.S. Pat. No. 6,333,311, which is incorporated herein by reference.

Substitutional variants or replacement variants typically contain the exchange of one amino acid for another at one or more sites within the protein. Substitutions can be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, e.g., still obtain a biological functionally equivalent protein. In making such changes, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

Still further, it is understood that an amino acid can be substituted for another having a similar hydrophilicity value and still obtains a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

Thus, in the present invention, substitutional variants or replacement can be produced using standard mutagenesis techniques, for example, site-directed mutagenesis as disclosed in U.S. Pat. Nos. 5,220,007; 5,284,760; 5,354,670; 5,366,878; 5,389,514; 5,635,377; 5,789,166, and 6,333,311, which are incorporated herein by reference. It is envisioned that at least the N-terminal glycine amino acid residue can be replaced or substituted with any of the twenty natural occurring amino acids, for example a positively charged amino acid (arginine, lysine, or histidine), a neutral amino acid (alanine, asparagine, cysteine, glutamine, glycine, isoleucine, leucine, methionine, phenylaline, proline, serine, threonine, tryptophan, tyrosine, valine) and/or a negatively charged amino acid (aspartic acid or glutamic acid). Still further, it is contemplated that any amino acid residue within the range of N1 to N16 can be replaced or substituted. It is envisioned that at least up to 16 of the N-terminal amino acids residues can be replaced or substituted as long as the protein retains it biological and/or functional activity, which is stimulating the production of various cytokines (e.g., IL-18, MIP-3α, GM-CSF or IFN-γ), by inhibiting various cytokines, (e.g., IL-2, IL-4, IL-5, IL-6, IL-10, or TNF-α), by modulating neutropenia in immunosuppressed subjects or modulating infection and/or inflammation in immunosuppressed subjects or modulating mucositis. Thus, the N-terminal lactoferrin variants of the present invention are considered functional equivalents of lactoferrin.

In terms of functional equivalents, it is well understood by the skilled artisan that, inherent in the definition of a “biologically functional equivalent” protein is the concept that there is a limit to the number of changes that may be made within a defined portion of the molecule while retaining a molecule with an acceptable level of equivalent biological activity and/or enhancing the biological activity of the lactoferrin molecule. Biologically functional equivalents are thus defined herein as those proteins in which selected amino acids (or codons) may be substituted. Functional activity is defined as the ability of lactoferrin to stimulate or inhibit various cytokines or chemokines and/or attenuate or modulate or inhibit or prevent neutropenia in immunosuppressed subjects and/or attenuate, modulate and/or prevent infection and/or inflammation in immunosuppressed subjects or attenuate, prevent or modulated mucositis.

Yet further, the lactoferrin polypeptides of this invention can be in glycosylated or unglycosylated form, can be modified post-translationally (e.g., acetylation, and phosphorylation) or can be modified synthetically (e.g., the attachment of a labeling group). Fragments of lactoferrin molecules that retain the prophylactic efficacy can also be used (see, e.g. PCT/IB00/00271, which is incorporated herein in reference in its entirety).

Still further, the N-terminal amino acid residues can be substituted with a modified and/or unusual amino acids. A table of exemplary, but not limiting, modified and/or unusual amino acids is provided herein below.

TABLE 1
Modified and/or Unusual Amino Acids

| Abbr. | Amino Acid |
|---|---|
| Aad | 2-Aminoadipic acid |
| BAad | 3-Aminoadipic acid |
| BAla | beta-alanine, beta-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| BAib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

The presence and the relative proportion of an N-terminal lactoferrin variants (deletions and/or substitutions) in a preparation of lactoferrin (lactoferrin composition) may be done by determination of the N-terminal amino acid sequence by the process of Edman degradation using standard methods. A relative proportion of N-terminal lactoferrin variant comprises at least 1% of the lactoferrin composition, at least 5% of the lactoferrin composition, at least 10% of the lactoferrin composition, at least 25% of the lactoferrin composition, at least 50% of the lactoferrin composition or any range in between.

In this method, the protein is reacted with phenylisothiocyanate (PITC), which reacts with the amino acid residue at the amino terminus under basic conditions to form a phenylthiocarbamyl derivative (PTC-protein). Trifluoroacetic acid then cleaves off the first amino acid as its anilinothialinone derivative (ATZ-amino acid) and leaves the new amino terminus for the next degradation cycle.

The percentage of N-terminal lactoferrin variant may also be done more precisely by using a Dansylation reaction. Briefly, protein is dansylated using Dansyl chloride reacted with the protein in alkaline conditions (pH 10). Following the Dansylation, the reaction mixtures are dried to pellets, then completely hydrolyzed in 6N HCl. The proportion of N-terminal amino acids are identified by RP HPLC using an in-line fluorometer in comparison with standards made up of known dansylated amino acids.

III. Pharmaceutical Compositions

The present invention is drawn to a composition comprising a lactoferrin composition that is dispersed in a pharmaceutical carrier. The lactoferrin that is contained in the composition of the present invention comprises lactoferrin or an N-terminal lactoferrin variant in which at least the N−1 terminal glycine residue is truncated or substituted. More specifically, the N-terminal lactoferrin variant comprises at least 1% of the composition, at least 5% of the composition, at least 10% of the composition, at least 25% of the composition, at least 50% of the composition or any range in between.

Further in accordance with the present invention, the composition of the present invention suitable for administration is provided in a pharmaceutically acceptable carrier with or without an inert diluent. The carrier should be assimilable and includes liquid, semi-solid, e.g., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof.

In accordance with the present invention, the composition is combined with the carrier in any convenient and practical manner, e.g., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, e.g., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc., proteolytic enzyme inhibitors, and the like. Yet further, it is envisioned that divalent metal chelators, for example EDTA, can also be used to stabilize the composition of the present invention. More preferably, for an orally administered composition, the stabilizer can also include antagonists to the secretion of stomach acids.

The composition for oral administration which is combined with a semi-solid or solid carrier can be further formulated into hard or soft shell gelatin capsules, tablets, or pills. More preferably, gelatin capsules, tablets, or pills are enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the lactoferrin composition to be released and absorbed by specialized cells, e.g., epithelial enterocytes and Peyer's patch M cells.

In another embodiment, a powdered composition is combined with a liquid carrier such as, e.g., water or a saline solution, with or without a stabilizing agent.

The amount of lactoferrin in the present invention may vary from about 1 g to about 100 g of lactoferrin. The lactoferrin may comprise lactoferrin or an N-terminal lactoferrin variant in which at least the N-1 terminal glycine residue is truncated and/or substituted.

In certain embodiments, an exemplary effective dose ranges between about 10 mg to about 70 g per day, preferably between about 0.5 to about 15 g per day, and one preferably 4.50 g per day. The 4.5 g daily dose (1.5 g×3) is expected to be well-tolerated.

Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug-release capsules and the like. Some variation in dosage can occur depending on the condition of the subject being treated. The person responsible for administration can, in any event, determine the appropriate dose for the individual subject.

IV. Treatment

In order to carry out methods of the present invention, a lactoferrin product is administered to an immunosuppressed individual or an individual whose immune system is expected to be suppressed.

As noted above, the subject method is suited for preventing infection and/or inflammation in immunosuppressed individuals or individuals whose immune systems are expected to be repressed. In one aspect, the immunosuppressed subject exhibits neutropenia, or suffers from AIDS or any form of cancer including lymphoma and myeloma. In yet another aspect, the subject undergoes or is expected to undergo a therapy selected from the group consisting of chemotherapy, radiotherapy, and autologous peripheral stem cell transplant.

An effective amount of the lactoferrin composition depends on the severity and/or course of immunosuppression, the patient's clinical history and response, and the discretion of the attending physician. The composition is suitably administered to the patient at one time or over a series of treatments. The initial candidate dosage may be administered to a patient. The proper dosage and treatment regimen can be established by monitoring the progress of therapy using conventional techniques known to the people skilled of the art.

The amount of active ingredients that may be combined with the carrier materials to produce a single dosage form will vary depending upon the subject treated and the particular mode of administration. It will be understood, however, that the specific dose level for any particular patient will depend upon a variety of factors, including the activity of the specific composition employed, the age, body weight, general health, sex, diet, time of administration, route of administration, and rate of excretion, drug combination and the severity of the particular disease undergoing therapy, and can be determined by those skilled in the art.

In order to achieve effective prophylaxis, preferably, the lactoferrin composition is administered prior to the inception neutropenia, more specifically Grade 3 or higher neutropenia and continued on the same schedule daily for the entire period during which the patient is at a high risk of neutropenic fever, infection or mucositis.

It is envisioned that the immune response is enhanced by lactoferrin stimulating cytokines and/or chemokines. Exemplary cytokines include interleukin-18 and GM-CSF in the gastrointestinal tract, which are known to enhance immune cells or stimulate production of immune cells. For example, interleukin-18 enhances natural killer cells or T lymphocytes. In specific embodiments, interleukin-18 (IL-18) enhances CD4+, CD8+ and CD3+ cells. It is known by those of skill in the art that IL-18 is a Th1 cytokine that acts in synergy with interleukin-12 and interleukin-2 in the stimulation of lymphocyte IFN-gamma production. Other cytokines or chemokines may also be enhanced for example, but not limited to IL-12, IL-1b, MIP-3α, MIP-1α or IFN-γ. Other cytokines or enzymes may be inhibited for example, but not limited to IL-2, IL-4, IL-5, IL-6, IL-10, TNF-α, or matrix metalloproteinases. It is further contemplated that lactoferrin inhibits the production of TNF-α, which inhibits cells involved in inflammation. It is also envisioned that lactoferrin stimulates interleukin-18 and a Th1 response following administration (e.g., oral), which inhibits pro-inflammatory cytokines, e.g., IL-4, IL-5, IL-6, IL-8 and TNF-α.

The lactoferrin composition of the present invention can also result in inhibition of a cytokine or chemokine. The cytokines include, but are not limited to interleukin-2 (IL-2), interleukin-4 (IL-4), interleukin-5 (IL-5), interleukin-10 (IL-10), and tumor necrosis factor alpha (TNF-α). Still further, the lactoferrin composition can also inhibit the production of matrix metalloproteinases (MMPs).

In further embodiments, cytokines, for example, interleukin-18 or granulocyte/macrophage colony-stimulating factor, can stimulate the production or activity of immune cells. The immune cells include, but are not limited to T lymphocytes, natural killer cells, NK-T cells, macrophages, dendritic cells, and polymorphonuclear cells. More specifically, the polymorphonuclear cells are neutrophils and the T lymphocytes are selected from the group consisting of CD4+, CD8+ and CD3+ T cells.

Certain treatment regimens include administering a lactoferrin composition in which the concentration of lactoferrin that is provided is in the range of 1 g to about 100 g/ml and the composition is a solution that is swished in the mouths of the subjects for a few minutes (e.g., about 4 minutes) and then swallowed. This process can be repeated mutiple times a day, for example twice or more each day, for a total of three doses per 24 hour period. The doses are taken immediately after meals (one each after breakfast, lunch and dinner), or at similarly spaced intervals throughout the day. Subjects are asked not to eat or drink anything for a least one hour after swallowing the dose. The treatment may be terminated in the event of significant drug related adverse effects observed during one of the interim safety analyses.

The multiple administration of lactoferrin each day can maximize the exposure of the oral cavity and gut to the drug. Preferably, patients receive 1.5 g LF three times per day prophylactically for 21-28 days starting from the commencement of chemotherapy. Daily dosing continues through the neutropenic phase until the earliest of the following: a) Absolute Neutrophil Count (ANC) is ≧600 cells/μL for three consecutive days, and mucositis (if present) has improved to Grade 2 or better for three consecutive days; or b) patient is discharged from the hospital or outpatient transplant setting. A last-day-of-therapy evaluation is performed within 24 hours after the last dose. The patients are also required to return for an end-of-treatment evaluation, to be performed 3-7 days after the last dose.

The treatment regimen in the present invention can prevent or attenuate infections in immunosuppressed patients, for example, patients undergoing aggressive chemotherapy or radiotherapy. The treatment reduces incidence, duration, and severity, and prolongs the infection-free interval of neutropenic infection (neutropenic fever). For the purpose of this invention, fever is considered as an oral temperature of at least 38.0° C. on at least two occasions within 24 hours, or a single oral temperature of at least 38.3° C. Neutropenic fever is defined as fever that begins when the patient is neutropenic (ANC<500). The end of neutropenic fever is defined as the point at which the patient begins a period of 48 hours without fever (as defined above), whether or not the patient is still neutropenic. Incidence of neutropenic fever is calculated as the number of patients experiencing one or more episodes of neutropenic fever. Onset of neutropenic fever is determined in two ways: (1) days between the transfusion and the first episode of neutropenic fever and (2) days between a neutrophil count <1000/uL and the first episode of neutropenic fever. Duration of neutropenic fever is defined as the number of days between the onset and the end of neutropenic fever. Severity of neutropenic fever is determined by the average temperature of the patient during the period of neutropenic fever. Infection-free interval is the period between the end of an infective fever and the onset of the next infective fever.

The present treatment also reduces the incidence, severity, and duration of oral and GI mucositis by improving all clinical criteria used for mucositis evaluation (pain, salivation, appetite, and oral inflammation). The incidence, duration, and severity of oral mucositis are assessed for each patient. Oral mucositis is graded using the Oral Mucositis Assessment Scale (OMAS).

Prophylactic and concomitant use of antibiotics may be used during the treatment according to patient's conditions and usual standard of care in the medical profession. Use of hematopoietic growth factors is not recommended routinely, but G-CSF may be used as part of the standard of clinical carte at the discretion of the doctors. All medications necessary for the patient's well being may also be administered.

The responses of the patients in the course of treatment are closely monitored by evaluating various response parameters (see Examples). The treatment protocol may be adjusted accordingly depending on the improvement of the patient's physical conditions.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Use of rhLF in Stem Cell Recipients

This example describes a prophylactic administration of rhLF in patients receiving autologous bone marrow transplants to prevent or reduce the incidence, duration, and severity of neutropenia and/or mucositis in this patient population.

Patients enrolled were 18 years or older who were receiving high dose chemotherapy prior to autologous bone marrow transplant, and expected to have a >50% incidence of grade ¾ neutropenia, and >40% incidence of grade ¾ oral mucositis. Patients were treated prophylactically with rhLF or placebo.

The dosing regime for rhLF or vehicle was begun via oral administration within 24 hours of the initiation of the conditioning regimen and continued for up to 28 days. RHLF was provided as a 100 mg/mL solution in a phosphate buffer (pH=7.0+0.1), in individual 15 mL unit doses. One vial (1.5 g of rhLF or a vehicle control) was administered 3 times a day at least 1 hour before meals or other oral intake, for a total of 4.5 g per day. Each dose was swished orally for 4 minutes to enhance buccal exposure before being swallowed. Placebo consisted of the identical phosphate buffer alone and was administered using the same regimen.

Mucositis endpoints included pain scores. Each patient graded pain on a scale from 1 to 10 including estimates for Throat and Abdominal Pain. Severity of mucositis was based on the maximal score graded by the patient in each of the above areas.

A total of 50 treated patients were evaluable for analysis (26 placebo and 24 rhLF). Results are shown in Table 2

TABLE 2

VI. Mean Maximum
Pain During Study

| | Throat Pain | Abdominal Pain |
|---|---|---|
| Placebo | 5.04 | 2.85 |
| RhLF | 3.96 | 2.54 |
| % Improvement | 21% | 11% |

### Example 2

### Use of rhLF in Myelosuppressive Chemotherapy

This example describes a the use of rhLF in patients undergoing myelosuppressive chemotherapy.

Adult patients who experience neutropenic fever (temperature>38.3 with ANC<500/mm³) in a previous cycle of chemotherapy and are to receive the same regimen at the same dose in the next cycle selected to receive lactoferrin treatment.

Briefly, four cohorts are recruited, with two placebo and eight actively treated subjects in each cohort. Each cohort receives 0.5 g, 1.0 g., 1.5 g or 2.0 g drug (or placebo) tid for a total daily dose of 1.5 g, 3 g, 4.5 g or 6 g rhLF. Administration of rhLF or placebo starts the day after the completion of chemotherapy and continue through the neutropenic phase until an absolute neutrophil count of 1,500/mm³ is reached or exceeded for two consecutive days.

Clinical parameters that are tested include, physical examination (including vital signs, height, weight); temperature (three times a day); patient questionnaire for oral and GI pain and dysphagia and objective determination of oral mucositis (incidence and grade). Laboratory parameters that are tested include hematology (e.g., clinical chemistry, urinalysis), absolute neutrophil count (ANC measured three times a week until ANC<1,000/mm³ and daily thereafter until recovery of counts), blood culture (BC is measured at baseline and on occurrence of neutropenic fever), serum and urine levels (measured rhLF and measurement of anti-rhLF antibodies at the time of the baseline visit, one day after initiation of rhLF, and within 24 hours of the completion of rhLF administration) and optional pharmacokinetic and anti-bacterial measurement of oral rhLF (through collection of oral swabs every 30 minutes for 3 hours following the first dose of rhLF). Other measurements can include incidence, duration and severity of oral mucositis by subjective (patient questionnaire) and objective (OMAS scale) criteria and incidence and duration of rehospitalization or visits to the Emergency Room.

### Example 3

### Use of rhLF to Treat Mucositis

This example describes the use of rhLF in reducing the incidence of patients developing mucositis as a result of aggressive radiation therapy for the treatment of cancer.

Four cohorts are recruited, with two placebos and eight actively treated subjects in each cohort. Cohorts receives 0.5 g, 1.0 g, 1.5 g or 2.0 g drug (or placebo) tid (3 times a day) for a total daily dose of 1.5 g, 3 g, 4.5 g or 6 g rhLF. Administration of rhLF or placebo starts the day after the completion of radiation therapy and continue for 21 days.

Patients are asked to swish the rhLF or placebo solution in their mouths for 4 minutes to coat the buccal mucosa, gargle and swallow the liquid preparation three times a day.

Primary endpoints are incidence, duration and severity of oral mucositis by subjective (patient questionnaire) and objective (OMAS scale) criteria. Patients are monitored according to standard clinical and laboratory parameters including physical, vital signs and blood analyses.

### Example 4

### Use of rhLF in AIDS Patients

This example describes the used of rhLF in AIDS patients who develop concomitant infections as a result of immune suppression stemming from HIV infection, for example reducing the incidence and severity of concomitant infections when administered to patients with HIV-1. Such opportunistic infections stem from suppression of the immune system by HIV and can include infections by bacteria, viruses, fungi, parasites and other microbes.

Patients recruited are diagnosed with HIV-1 and have an AIDs defining illness or a CD4+ cell counts lower than 200 per cubic millimeter.

Patients are divided into four cohorts with five placebo and fifteen actively treated subjects in each cohort. Cohorts receive 0.5 g, 1.0 g or 2.0 drug (or placebo) tid for a total daily dose of 1.5 g, 3 g or 6 g rhLF over a total period of 30 days.

For patients infected with HIV, standard clinical end points are either distant in time or rare, and thus require a trial of long duration, large sample size, or both to demonstrate the value of a new treatment. A surrogate marker for traditional clinical end points is needed to allow more rapid completion of clinical investigations of new therapies. In this trial primary endpoints are incidence, duration and severity of concomitant infection, changes in HIV-1 RNA plasma levels, changes in CD4+ cell counts and levels of Serum p24 antigen which is regarded as earliest serologic marker specific for HIV infection. Progression to A/DS and mortality are used as secondary endpoints.

Additional efficacy endpoints include incidence, duration and severity of the symptoms of opportunistic infection including: coughing and shortness of breath, seizures and lack of coordination, difficult or painful swallowing, mental symptoms such as confusion and forgetfulness, severe and persistent diarrhea, fever, vision loss, nausea, abdominal cramps, and vomiting, weight loss and extreme fatigue, severe headaches, and coma.

HIV positive children enrolled on the study are evaluated separately using all of the efficacy criteria mentioned earlier plus severe bacterial infections seen particularly in children with A/DS including conjunctivitis, ear infections and tonsillitis.

### Example 5

### Prophylaxis of Infection and Inflammation in Patients Receiving Oral Corticosteroids

This example describes the use of rhLF in patients who are at risk of developing infections as a result of immune suppression stemming from large doses of oral corticosteroids. The study determines the efficacy of various doses of rhLF in reducing the incidence, severity and duration of infections, the incidence, severity and duration of GI ulceration and incidence and duration of hospitalization and other medical interventions in these patients.

Patients are recruited who are receiving therapy with oral corticosteroids and are at risk of becoming immunosuppressed and developing opportunistic infections and GI ulceration.

Patients are divided into four cohorts with five placebo and fifteen actively treated subjects in each cohort. Cohorts receive 0.5 g, 1.0 or 2.0 g drug (or placebo) tid for a total daily dose of 1.5 g, 3 g or 6 g rhLF over a total period of 30 days.

Primary endpoints are incidence, duration and severity of infection and incidence, severity and duration of GI ulceration, and incidence and duration of hospitalization and other medical interventions.

### Example 6

### Effect of Oral rhLF in Candida tropicalis-Infected Severely Neutropenic Mice

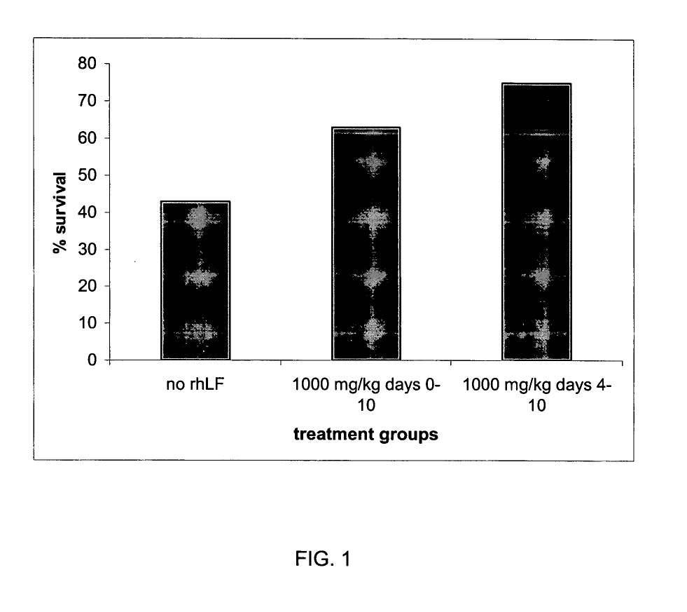

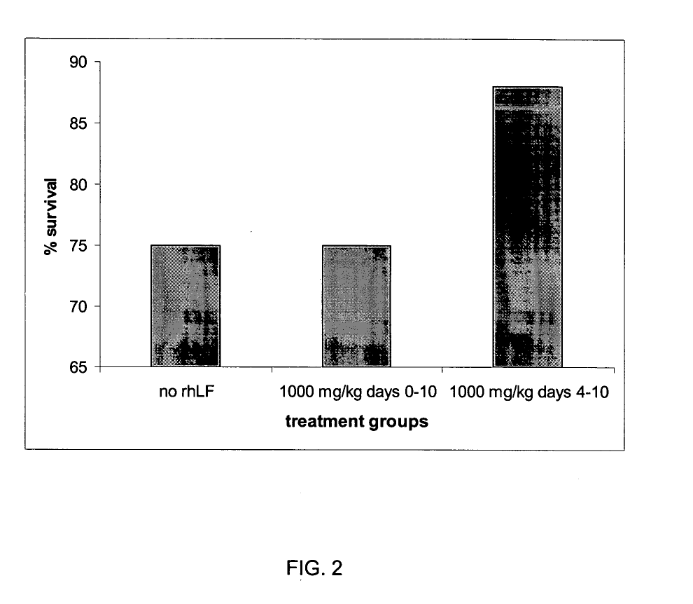

CD-1 (ICR), 5-6 weeks old mice, 22-25 g body weight were used. Mice were administered cyclophosphamide (200 mg/kg) by i.p. injection on days 1 and 3 of the experiment. Using separate control animals, the absolute neutrophil count (ANC) was measured on days 4, 5, 6, and 7, confirming that mice developed severe neutropenia (ANC<100 neutrophils/mm³) by Day 4. Neutropenic mice were challenged on Day 5 with either a high (1.5×10⁶ CFU/mL) or a low (1.5×10⁵ CFU/mL) dose of Candida tropicalis (FIGS. 1 and 2 respectively).

Mice were treated orally with 1000 mg/kg/day rhLF either from Day 0 to Day 10, or from Day 4 through to Day 10. Mortality was recorded until Day 10 of the experiment. Results with rhLF-treated mice were compared to neutropenic mice challenged with Candida tropicalis but receiving vehicle control instead of the test compound.

FIGS. 1 and 2 show that treatment with rhLF increased the survival of severely neutropenic mice infected with Candida tropicalis.

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Pat. No. 5,789,166
U.S. Pat. No. 5,635,377
U.S. Pat. No. 5,629,001
U.S. Pat. No. 5,571,896
U.S. Pat. No. 5,571,697
U.S. Pat. No. 5,571,691
U.S. Pat. No. 5,389,514
U.S. Pat. No. 5,366,878
U.S. Pat. No. 5,354,670
U.S. Pat. No. 5,284,760
U.S. Pat. No. 5,220,007
U.S. Pat. No. 6,333,311
Arnold et al., Infect Immun. 28:893-8 (1980)
Bellamy et al., Med. Microbiol. Immunol. 182(2): 97-105 (1993)
Cumberbatch et al., Immunology 100(1):21-8 (2000)
Dial et al., Biochem. Cell. Biol. 80:113-7 (2002);
Ellison et al., Infect Immun. 56(11):2774-81 (1998)
Harsen et al., J. Infect. Dis. 172 (2):380-8 (1995)
Ikeda et al., Biochem. Biophys. Res. Commun. 245(2): 549-53 (1999)
Inoue et al., Bone Marrow Transplant: 22(4) 402-3 (1998)
Inoue et al., 5th Annual Conference on Lactoferrin, Bamff, Canada (2001)
Isamida et al., J. Vet. Med. Sci. 60(2):241-4 (1998)
Kimber et al., Lactoferrin: Structure, function, and Applications. Elsevier Science B.V., 87-93 (2000)
Levay et al., Haematologica. 80: (3):252-67 (1993)
Levay et al., Haematologica. 80(3):252-67 (1995)
Marchetti et al., Biometals. 11(2):89-94 (1998)
Masson et al., Comp. Biochem. Physiol. B. 39(1):119-29 (1971);
Matthews et al., Lancet. 2(8000): 1387-9 (1976)
Metz-Boutigue et al., Eur. J. Biochem. 145(3):659-76 (1984);
Miehlke et al., Helicobacter pylori. J. Clin. Microbiol. 34(10): 2593-4 (1996)
Sato et al., AJVR 57(10):1443-1446 (1996)
Tomita J. Dairy Sci. 74 (12): 4137-42 (1991
Trumpler et al., Eur. J. Clin. Microbiol. Infect. Dis. 8: (4):310-313 (1989)
Vorland et al., APMIS 107:971-81 (1999)
Ward et al., Biotechnology 13(5): 498-503 (1995)

Although the present invention and its advantages have been described in detail, it should be understood that various changes, substitutions and alterations can be made herein without departing from the spirit and scope of the invention as defined by the appended description. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended descriptions are intended to include within their scope such processes, machines, manufacture, compositions of matter, means, methods, or steps.

1. A method for modulating an infection and/or inflammation in an immunosuppressed subject suffering from mucositis, comprising administering to the subject an effective amount of a lactoferrin composition, wherein the amount reduces the infection and/or inflammation in said subject.

2. The method of claim 1, wherein the immunosuppressed subject undergoes a therapy selected from the group consisting of chemotherapy, radiotherapy, myelosuppressive therapy, and myeloablative therapy.

3. The method of claim 1, wherein the immunosuppressed subject is a cancer patient undergoing stem cell or bone marrow transplantation.

4. The method of claim 2, wherein said subject suffers from a hematopoietic cancer or a solid tumor malignancy.

5. The method of claim 2, wherein the said subject suffers from a cancer selected from the group consisting of lymphoma, myeloma, melanoma, non-small cell lung cancer, small-cell lung cancer, hepatocarcinoma, retinoblastoma, astrocytoma, gliobastoma, leukemias, neuroblastoma, squamous cell cancer, head cancer, neck cancer, gum cancer, tongue cancer, breast cancer, pancreatic cancer, prostate cancer, renal cancer, bone cancer, testicular cancer, ovarian cancer, mesothelioma, sarcoma, cervical cancer, gastrointestinal cancer, brain cancer, colon cancer, myelodysplastic syndrome, and bladder cancer.

6. The method of claim 1, wherein the immunosuppressed subject is infected with HIV.

7. The method of claim 1, wherein the subject has received or is receiving an immunosuppressive agent.

8. The method of claim 7, wherein the immunosuppressive agent is corticosteroids.

9. The method of claim 1, wherein the composition is administered orally, intravenously, intramuscularly, subcutaneously or transdermally.

10. The method of claim 1, wherein said effective amount is from about 10 mg lactoferrin per day to about 70 gram lactoferrin per day.

11. The method of claim 1, wherein said lactoferrin composition is dispersed in a pharmaceutically acceptable carrier.

12. The method of claim 1, wherein said lactoferrin is recombinant lactoferrin.

13. The method of claim 1, wherein said lactoferrin composition comprises an N-terminal lactoferrin variant.

14. The method of claim 1, further comprising administering an antibiotic, growth factor or a combination thereof.

15. A method for prophylactically treating neutropenia in a subject who is expected to undergo a therapy that results in reduction of neutrophils in the subject, comprising administering to the subject an effective amount of a lactoferrin composition prior to the therapy, or development of neutropenia, wherein said amount increases the levels of neutrophils in the subject.

16. The method of claim 15, wherein the subject is infected with HIV.

17. The method of claim 15, wherein the subject has received or is an immunosuppressive agent.

18. The method of claim 17, wherein the immunosuppressive agent is corticosteroids.

19. A method of prophylactically treating an infection and/or inflammation in an immunosuppressed subject comprising administering to the subject a prophylactic amount of a lactoferrin composition, wherein the amount modulates the infection and/or inflammation by reducing mucositis and/or increasing the neutrophil count in the subject.
